(19) United States
(12) Patent Application Publication
(10) Pub. No.: US 2007/0161072 A1
(43) Pub. Date: Jul. 12, 2007

(54) Neuroprotection by inhibition of diacyglycerol kinase epsilon activity

(76) Inventors: Stephen M. Prescott, Solt Lake City, UT (US); Matthew Topham, Salt Lake City, UT (US); Nicolas G. Bazan, New Orleans, LA (US); Elena B. Rodriguez de Turco, New Orleans, LA (US)

Correspondence Address: MADSON & AUSTIN;GATEWAY TOWER WEST, SUITE 900, 15 WEST SOUTH TEMPLE, SALT LAKE CITY, UT, 84101, US

(21) Appl. No.: 11/710,067

(22) Filed: Feb. 23, 2007

Related U.S. Application Data
(62) Division of application No. 10/471,116, filed on Sep. 8, 2003, now abandoned.
(60) Provisional application No. 60/277,917, filed on Mar. 22, 2001.

(51) Int. Cl. A61K 31/66; C12Q 1/48

(52) U.S. Cl. 435/15; 514/102

(57) ABSTRACT

The present invention includes the characterization of the DGKε gene and the generation of screening methods for compounds that inhibit the function of DGKε. The DGK family of enzymes occupies a signaling crossroads since they catalyze the phosphorylation of DAG to produce PA. Both the substrate (DAG) and the product (PA) of this reaction are key factors in intracellular signaling, making the regulation of DGKε activity important to understand and control. DGKε −/− mice were also generated and studied to assist in understanding the function of DGKs in regulating cellular signaling. DGKε displays selectively for 20:4-DAG and is highly expressed in different areas of the brain, including Purkinje cells in the cerebellum, hippocampal interneurons, and the Pyramidal neurons in the CA3 region of the hippocampus.

### CROSS-REFERENCED RELATED APPLICATIONS

This application is a divisional of prior application Ser. No. 10/471,116, filed Sep. 8, 2003, which was the National Stage of International Application No. PCT/US02/08853, filed Mar. 22, 2002, which claims the benefit of U.S. Provisional Application No. 60/277,917, filed Mar. 22, 2001, which is incorporated herein by reference.

### BACKGROUND OF THE INVENTION

The present invention relates to kinases involved in cellular signaling and synaptic function. Specifically, the present invention relates to diacylglycerol kinase epsilon and its role in the modulation of multiple neuronal signaling pathways linked to synaptic activity, neuronal plasticity, and epileptogenesis.

### TECHNICAL BACKGROUND

Diacylglycerol (or “DAG”) is an important chemical signal which functions in several intracellular signaling pathways. One of these is a pathway initiated by the hydrolysis of phosphatidylinositol 4,5-bisphosphate (“PIP2”). This reaction results in a transient rise in the amounts of diacylglycerol, a lipid messenger; and inositol 1,4,5-trisphosphate (“IP3”), a polar molecule. Rhee & Bae, J. Biol. Chem., 272, 15045-15048 (1997). The IP3 binds to intracellular receptors to initiate calcium release from intracellular stores, and the DAG functions as an allosteric activator of protein kinase C (“PKC”). Clapham, Cell, 80, 259-268 (1995); Newton, Curr. Opin. Cell Biol., 9, 161-167 (1997). The removal of DAG by diacylglycerol kinases (“DGKs”) is thought to attenuate these actions, so DGKs are thought to terminate the activity of PKCs and other DAG-activated proteins. The IP3 and the DAG are then both converted to inactive products, thus causing the cell to return to its basal state. Majerus et al., J. Biol. Chem., 274, 10669-10672 (1999).

In addition to activating PKC, DAG participates in other cellular events. It is a potent activator of the guanine nucleotide exchange factors vav and Ras-GRP, indicating a potential role for DAG in regulating Ras and Rho family proteins. Gulbins et al., Mol. Cell. Biol., 14, 4749-4758 (1994); Nishizuka, Science, 233, 305-312 (1986); Ebinu et al., Science, 280, 1082- (1988). In addition to these signaling roles, DAG occupies a central position in the synthesis of major phospholipids such as phosphatidylcholine and phosphatidylethanolamine; and of triacylglycerols. Carman & Zeimetz, J. Biol. Chem., 271, 13293-13296 (1996). Thus, to maintain cellular homeostasis, intracellular DAG levels must be tightly regulated.

Specific evidence supports the apparent importance of cellular DAG regulation. First, it has been observed that inappropriate accumulation of DAG by a cell contributes to cellular transformation. In one study, for example, cell lines that overexpress phospholipase C (here, phospholipase Cγ, or “PLCγ”) have a malignant phenotype. Chang et al., Cancer Res., 57, 5465-5468 (1997). In another, cells transformed with one of several oncogenes were observed to have elevated DAG levels, and it was seen that growth factors that are proto-oncogenes stimulate the PLC pathway. Kato et al., J. Biol. Chem., 262, 5696-5704 (1987); Kato et al., Biochem. Biophys. Res. Commun., 154, 959-966 (1988); Priess et al., J. Biol. Chem., 261, 8597-8600 (1986); Wolfman et al., J. Biol. Chem., 262, 16546-16552 (1987).

Most of the evidence explaining this pathological effect centers on the excessive and/or prolonged activation of PKC, which has been observed to be a common feature of the transformed state, both in tumors and in cell cultures. Housey et al., Cell, 52, 343-354 (1988). Indeed, PKC function was identified in part by virtue of its property of being the target for phorbol esters. Phorbol esters are tumor promoters that function in the same way as DAG to activate PKC, but differ from DAG in that they persist. This persistence appears to be due to either very slow or nonexistent metabolism of the ester molecules. These observations have led to the hypothesis that prolonged elevation of DAG functions as a tumor promoter—the equivalent of an endogenous phorbol ester.

Diacylglycerol kinases are a family of enzymes that regulate the levels of DAG. Specifically, DGKs phosphorylate diacylglycerol to phosphatidic acid (“PA”). PA is a signaling molecule that stimulates DNA synthesis and modulates the activity of several enzymes including phosphatidylinositol 5-kinases (“PI-5-K”). Rameh & Cantley, PAK1, PKCζ, and Ras-GAP; Knauss et al., J. Biol. Chem., 265, 14457-14463 (1990); van Corven et al., Biochem. J., 281, 163-169 (1992); Bokoch et al., J. Biol. Chem., 273, 8137-8144 (1998); Exton, Physiol. Rev., 77, 303-320 (1997); Rameh & Cantley, J. Biol. Chem., 274, 8347-8350 (1999). Although the bulk of the signaling “pool” of PA (which is also an intermediate in phospholipid synthesis) is thought to derive from the action of phospholipase D (“PLD”), DGKs likely contribute to it as well. Exton, Physiol. Rev., 77, 303-320 (1997). Thus, DGKs catalyze a reaction that removes DAG and would terminate the PKC-mediated signal but yield a product, PA, which has other functions in signaling and phospholipid synthesis. PA has been reported to modulate atypical PKC isoforms, Ras-GAP, phosphatidylinositol (“PI”) 5-kinases, and other signaling proteins, and PA is a mitogen for a variety of cells. Moritz et al., 1992; Exton, Physiological Reviews, 77, 303 (1997).

It is unclear which functions attributable to DAG and PA reflect the actions of DGK, since PLD also releases PA, and DAG is also produced by PA phosphatase. Sakane & Kanoh, Int. J. Biochem. Cell. Biol., 29, 1139-1143 (1997); Topham & Prescott, J. Biol. Chem., 274, 11447-50 (1997); Exton, Physiological Reviews, 77, 303- (1997). It is likely, however, that signaling lipids derived from each pathway—the PLC/DGK pathway or the PLD/PA phosphatase pathway—have distinct functions by virtue of the parent lipids for each reaction. Hodgkin et al., Trends Biochem. Sci., 23, 200- (1998). For example, the predominant substrate of PLD is phosphatidylcholine, which contains primarily mono unsaturated fatty acids at the C2 position of glycerol backbone, so the reaction product, phosphatidic acid, is also enriched with mono unsaturated fatty acids. Trends Biochem. Sci., 23, 200- (1998).

Alternatively, DGKs are thought to phosphorylate DAG generated by PI-specific PLCs. Since inositol phospholipids are enriched in the polyunsaturated fatty acid arachidonic acid (20:4n-6, AA) at the C2 position, DAG derived from this reaction is predominantly AA-DAG, so the PA generated by DGK activity also displays high content of the polyunsaturated fatty acid AA. Trends Biochem. Sci., 23, 200- (1998). And, there is evidence that DAG and PA, depending on their lipid composition, can differentially activate protein targets. For example, unsaturated DAG has been shown to be a more potent activator of protein kinase Cs than saturated DAG, while saturated PA species induce MAPK activation to a greater extent that unsaturated PAs. Thus, DGKs and PLDs likely influence distinct signaling events. Trends Biochem. Sci., 23, 200- (1998).

While most attention on PA signaling has been focused on the “PLD” reaction, PA generated by DGKs likely has signaling functions as well. Recent research identified a potential role for DGK-generated PA in T lymphocyte proliferation. Flores et al., J. Biol. Chem., 271, 10334-10340 (1996). That study also noted that when T lymphocytes were treated with L-2, which is a growth signal, DGKα translocates to the perinuclear space. By using DGK inhibitors, data was generated that constituted evidence that the PA produced by this isozyme is necessary for progression to S phase of the cell cycle. This suggests that the PA generated by DGKα in this context had a signaling role.

In another study, active DGKα was demonstrated to be required for hepatocyte growth factor-induced migration of endothelial cells. Cutrupi et al., EMBO J., 19, 4614-4622 (2000). The data from this study suggested that generation of PA by DGKα is necessary for the migration, but the protein target of the phosphatidic acid could not be identified.

As mentioned above, there are many proteins whose activity can be influenced by PA, so DGKs could regulate a variety of cellular events that are dependent on PA. Diacylglycerol kinases can also influence proteins regulated by DAG. DGKs are likely inhibitory, however, because they terminate DAG signaling. Indeed, it has recently been demonstrated that DGKζ, and not other DGK isotypes, inhibits the activity of RasGRP, a Ras guanyl nucleotide exchange factor (“GEF”) whose activity requires DAG. Topham & Prescott, J. Cell Biol., 152 (in press) (2001). Additionally, another study presented evidence that a Caenorhabditis elegans DGK negatively regulates synaptic transmission by metabolizing DAG that would otherwise activate Unc-13, a protein activated by DAG that participates in neurotransmitter secretion. Nurrish et al., Neuron, 24, 231-242 (1999). Thus, by virtue of their enzymatic activity, DGKs can influence signaling events mediated by both DAG and PA. The net effect on cellular events is difficult to predict, but all of the potential outcomes appear to support the conclusion that DGKs occupy a very interesting niche.

DGKs are a large and widely distributed family of enzymes seen in prokaryotes and eukaryotes alike. DGKs have been identified in bacteria, Drosophila melanogaster, Caenorhabditis elegans, and plants. Badola & Sanders, J. Biol. Chem., 272, 24172-24182 (1997); Harden et al., Biochem. J., 289, 439-444 (1993); Masai et al., Proc. Natl. Acad. Sci., U.S. A., 89, 6030-6034 (1992); Masai et al., Proc. Natl. Acad. Sci., U.S.A., 90, 11157-11161 (1993); Katagiri et al., Plant Mol. Biol., 30, 647-653 (1996). DGK from Escherichia coli has also been identified, and is the most known member of a family of prokaryotic DGKs that have little structural relationship to the eukaryotic DGK family of enzymes. Because there is no evidence that there is a signaling function for DAG in bacteria, the DGKs presumably serve exclusively for the synthesis of complex lipids.

The E. coli DGK has also established itself in a technological niche serving as a reagent to determine DAG levels. Priess et al., J. Biol. Chem., 261, 8597-8600 (1986). Mammalian DGK activities have been identified in multiple cell types and a wide range of tissues, indicating their functional significance. Kanoh et al., Trends Biochem. Sci., 15, 47-50 (1990).

As noted above, the DGK family of kinases is large and diverse. As with other enzymes in signaling pathways, such as PKC and PI-specific PLC, mammalian DGKs are a family whose isozymes differ in their structures, patterns of tissue expression and catalytic properties. Sakane & Kanoh, Int. J. Biochem. Cell. Biol., 29, 1139-1143 (1997); and Topham & Prescott, J. Biol. Chem., 274, 11447-50 (1997). Nine mammalian DGK isoforms have been identified. All of them contain a catalytic domain that is necessary for kinase activity. The DGK catalytic domains likely function similarly to the C3 regions of PKCs by presenting ATP as the phosphate donor. One interesting feature of DGKs δ and η (and one Drosophila DGK) is that their catalytic domains are bipartite, indicating that the two modules may act cooperatively. Masai et al., Proc. Natl. Acad. Sci., U.S.A., 89, 6030-6034 (1992). All of the DGK catalytic domains have at least one presumed ATP binding site with the consensus sequence GXGXXG that is also found in protein kinases. Hanks et al., Science, 241, 42-52 (1988).

Studies of this sequence show that a mutation of the third glycine to aspartate abolishes activity of DGKs ε, ζ, and of a Drosophila DGK. Clapham, D. E. (1995) Cell 80, 259-268 (1995); Masai et al., Proc. Natl. Acad. Sci., U.S.A., 90, 11157-11161 (1993). This ATP binding motif differs from that of the protein kinases, where there is an essential lysine 14-23 amino acids downstream of the glycines. Hanks et al., Science, 241, 42-52 (1988). All of the known DGKs have a lysine in a similar position, but site-directed mutagenesis of this lysine in DGKs α, ε, or ζ does not alter activity. This indicates, without being bound to any one theory, that the ATP binding pockets of DGKs likely have a different conformation than the protein kinases. Schaap et al., Biochem. J., 304, 661-664 (1994); Sakane et al., Biochem. J., 318, 583-590 (1996).

In addition to these domains, all DGKs have at least two cysteine-rich regions homologous to the C1A and C1B motifs of PKCs. DGKθ has three. These domains in DGKs are thought to present DAG for phosphorylation, but this has not been conclusively demonstrated. They may also bind phorbol esters.

However, C1 domains in several proteins clearly do not bind DAG and in some cases serve instead as sites of protein-protein interaction. Brtva et al., J. Biol. Chem., 270, 9809-9812 (1995). Sakane et al. observed that DGKα was still active without its C1 domains and that DGKs α, β, and γ all failed to bind phorbol esters. Sakane et al., Biochem. J., 318, 583-590 (1996). It has been observed, however, that DGKζ, when lacking either of its two C1 domains, is inactive.

Hurley et al. recently examined the sequence homology of 54 C1 domains, including those of six DGKs (α, β, γ, δ, ε, and ζ). Hurley et al., Protein Sci., 6, 477-480 (1997). It was proposed that except for the C1A domains of DGKs β and ζ, all other DGK C1 domains may not bind DAG. The different functions of the proteins must be considered, however, since it appears that when PKCs bind DAG, they essentially exclude it from an attacking phosphoryl group, which is sensible as the DAG is functioning as an allosteric activator. In contrast, DGKs must present DAG for phosphoryl transfer, which suggests that they might bind it differently; thus, an altered C1 conformation might serve such a purpose.

In addition to their catalytic and C1 domains, most DGKs have structural motifs that form the basis for dividing them into five subtypes and that likely play regulatory roles. Type I DGKs have calcium binding EF hand motifs at their N termini, making these isoforms calcium-responsive to slightly different extents. Sakane et al., Nature, 344, 345-348 (1990); Goto & Kondo, Proc. Natl. Acad. Sci., U.S.A., 90, 7598-7602 (1993); Kai et al., J. Biol. Chem., 269, 18492-18498 (1994); Yamada et al., Biochem. J., 321, 59-64 (1997). Diacylglycerol kinases having pleckstrin homology (“PH”) domains at their N termini are defined as type II DGKs. Sakane et al., J. Biol. Chem., 271, 8394-8401 (1996); Klauck et al., J. Biol. Chem., 271, 19781-19788 (1996). Takeuchi et al. found that the PH domain of DGKδ could bind “PI”. Takeuchi et al., Biochim. Biophys. Acta, 1359, 275-285 (1997). DGKB also has at its C terminus a region homologous to the EPH family of receptor tyrosine kinases. The function of this domain is unclear. No specific function has been identified for these domains. DGKδ also has at its C-terminus a sterile alpha motif (“SAM”). Its function is unclear, but SAM domains can be sites of protein-protein interactions.

DGKε is a type III enzyme, and although it does not have any identifiable regulatory domains, it strongly prefers an arachidonoyl group at the sn-2 position making it the only DGK that has specificity toward acyl chains of DAG. This preference suggests that DGKε may be a component of the PI cycle that accounts for the enrichment of PI species with arachidonate. DGKε has the simplest structure of the known DGKS since it has no identifiable regulatory domains. It is the sole member of group III DGKs to date. Tang et al., J. Biol. Chem., 271, 10237-10241 (1996).

Paradoxically, although DGKε is the simplest in structure, it is the only DGK known to have substrate specificity. DGKε strongly prefers DAG substrates with an arachidonoyl group at the sn-2 position over other substrates. This preference initially suggested that DGKε might be the component in the PI cycle that accounted for the enrichment of PI with arachidonate. Since, however, this isoform has a limited tissue distribution, even if this is the case, it cannot serve this function broadly. Prescott & Majerus, J. Biol. Chem., 256, 579-582 (1981).

Type IV DGKs have a region homologous to the phosphorylation site domain of the MARCKS protein, and at their C-termini, four ankyrin repeats. Bunting et al., J. Biol. Chem., 271, 10230-10236 (1996); Ding et al., J. Biol. Chem., 273, 32746-32752 (1998). One enzyme of this family, DGKζ, undergoes tissue-specific alternative splicing that results in an enzyme with an elongated N terminus; it is found predominantly in muscle. Ding et al., Proc. Natl. Acad. Sci., U.S.A., 94, 5519-5524 (1997). Both DGKζ and DGKι have a region homologous to the phosphorylation site domain of the MARCKS protein.

Finally, DGKθ defines group V. DGKθ has three cysteine-rich domains and a PH domain, as well as a region that is structurally similar to those in other proteins that have been implicated as mediating association with Ras, although this point is controversial. Houssa et al., J. Biol. Chem., 272, 10422-10428 (1997); Kalhammer et al., FEBS Lett., 414, 599-602 (1997). The complexity and diversity of the DGK family strongly suggest that the DGKs perform multiple roles in cellular functions.

Because DGKs influence cellular DAG and PA levels, control of their activity is essential; and this may be an important mechanism by which different functions are segregated. Specifically, DAG that is used for complex lipid synthesis may be a different pool than the one used as a signal. Also, the activation of Type I DGK activity by calcium represents a potentially elegant way to increase DGK activity in situations in which DAG is elevated. In fact, an increase in neuronal calcium upon stimulation (e.g. glutamate interaction with post-synaptyic NMDA receptors), activates the PLC signaling with the release of DAG and IP3. Bazan et al., J. Neurotrauma, 12, 791-814 (1995). The latter may contribute to a further increase in cytosolic calcium by mobilization from intracellular stores.

The DGK pathway will attenuate the DAG signal, returning that arm of the signaling cascade to a quiescent state. Other than this effect, however, relatively little is known about how the activity is regulated. Several compounds have been observed to modulate DGK activity in cellular homogenates, including: arachidonic acid, vitamin E, sphingosine, 15-hydroxyeicosatetraenoic acid, ceramide, and several fatty acids. Rao et al., J. Neurochemistry, 63, 1454-1459 (1994); Tran et al., Biochim. Biophys. Acta, 1212, 193-202 (1994); Sakane et al., FEBS Lett., 255, 409-413 (1989); Setty et al., J. Biol. Chem., 262, 17613-17622 (1987); Younes et al., J. Biol. Chem., 267, 842-847 (1992); Kelleher & Sun et al., J. Neurosci. Res., 23, 87-94 (1989); Vaidyanathan et al., Neurosci. Lett., 179, 171-174 (1994). One study observed that dietary fatty acids induce marked alterations in DGK activity in colon tumors. Reddy et al., Cancer Res., 56, 2314-2320 (1996). Another found that PIP2 is a potent inhibitor of arachidonoyl-specific DGK activity. Walsh et al., J. Biol. Chem., 270, 28647-28653 (1995). Several investigators have examined post-translational modifications that may regulate DGK function. Schaap and co-workers and Kanoh et al. have shown that DGKε is phosphorylated by PKC isoforms in vitro and in vivo. Schaap et al., Biochem. J., 289, 875-881 (1993). Kanoh et al., Biochem. J., 258, 455-462 (1989).

These findings were consistent with previous observations that suggested that PKC regulates the activity of DGK in cellular homogenates; however, a functional consequence resulting from the PKC-mediated phosphorylation has not yet been identified. Soling et al., J. Biol. Chem., 264, 10643-10648 (1989).

Understanding that DGKs occupy crucial positions in the regulation of cellular signaling agents, it would thus be an improvement in the art to clone and characterize the function of a DGK isozyme. Specifically, it would be an improvement in the art to clone and characterize the function of the murine DGKε enzyme. It would be a further improvement in the art to provide methods of screening for compounds which either upregulate or inhibit a DGK enzyme, such as the DGKε enzyme. It would also be an improvement in the art to provide methods for treating disorders selected from the group consisting of seizures, neurodegenerative disorders, and ischemic damage in a mammal using compounds found to inhibit the function of the DGKε gene product using the screening methods of the invention. Finally, it would be an improvement in the art to provide a transgenic nonhuman animal whose germ cells and somatic cells contain at least one chromosome comprising a disruption to the endogenous DGKε gene. It would be an improvement to provide such a transgenic nonhuman animal wherein the disruption to the endogenous DGKε gene results in a lack of expression of DGKε gene product. Such methods and transgenic nonhuman animals relating to DGKε are provided herein.

### BRIEF SUMMARY OF THE INVENTION

The present invention comprises in part the cloning and characterization of a murine DGKε. The enzyme was shown to display selectivity for 20:4-DAG. DGKε was further shown to be highly expressed in different areas of the brain, including Purkinje cells in the cerebellum, hippocampal interneurons, and the Pyramidal neurons in the CA3 region of the hippocampus. Mice with targeted disruption of the DGKε gene were also generated. Studies of these mice demonstrated that a DGKε deficiency affects multiple signaling pathways, including the PIP2-PLC and the cPLA2-20:4 pathways. This deficiency is further shown to yield higher resistance of neurons to seizures and attenuation of LTP as well as possible higher resistance to ischemic neuronal damage.

The invention first relates to methods of screening for potential agents for the regulation of DGKε activity. These methods include the steps of contacting a cell with a test compound, wherein the cell expresses or over-expresses DGKε, and measuring the level of DGKε activity in the cell. A test compound which increases or decreases the activity of DGKε in the cell is a potential agent that regulates DGKε activity. In this method, the DGKε activity to be measured may be the enzymatic conversion of 20:4-DAG 20:4-PA. Some of the potential agents that regulate DGKε activity do so by interfering with the binding of 20:4-DAG to DGKε. Further, the cell contacted with the test compound may be part of a multicellular organism. Alternatively, the cell may be derived from the brain, heart, retina, or testis of an organism.

The invention also includes methods of screening for potential agents for the regulation of DGKε activity. These methods include the steps of administering a test compound to an animal, administering a seizure stimulus to the animal, and measuring the level of DGKε activity in the animal in comparison with a control. A test compound which increases or decreases the activity of DGKε in the cell is a potential agent that regulates DGKε activity. In such methods, the activity of DGKε to be measured in the animal after the seizure stimulus is the enzymatic conversion of 20:4-DAG to 20:4-PA. In others, the activity of DGKε is measured by evaluating the level of PIP2 degradation compared to the control. Alternatively, the activity of DGKε is measured by evaluating the resistance of the animal to electroconvulsive shock. In still other alternatives, the activity of DGKε is measured by evaluating the attenuation of long term potentiation in the perforant path-dentate granular cell synapses of the animal. The seizure stimulus may include methods such as electroconvulsive shock, audiogenic stimuli, or the administration of proconvulsive pharmacological agents, or other methods known in the art of inducing a seizure.

The invention first includes methods of screening for potential agents for the treatment of disorders selected from the group consisting of seizures, neurodegenerative disorders, and ischemic damage. Herein, the term “stroke” includes status epileticus seizures often caused by head trauma, as well as epileptic seizures. Neurodegenerative disorders is a family of disease states that includes Alzheimer's disease and Parkinson's disease. Ischemic damage is a disorder often caused by stroke.

The methods comprise the step of contacting a cell with a test compound. The cell expresses or over-expresses a DGKε gene product. An additional step is measuring the inhibition of the function of the DGKε gene product in the cell. A test compound that inhibits the function of the DGKε gene product is a potential agent for treating disorders selected from the group consisting of seizures, neurodegenerative disorders, and ischemic damage.

According to such methods, the function of the DGKε gene product in the cell to be measured is the enzymatic conversion of 20:4-DAG 20:4-PA. In others, the test compound interferes with the binding of 20:4-DAG to DGKε. The cell may be part of a multicellular organism. Alternatively, the cell is derived from the brain, heart, retina, or testis of an organism.

The invention also comprises a method of screening for potential agents for treatment of disorders selected from the group consisting of seizures, neurodegenerative disorders, and ischemic damage, comprising the steps of administering a test compound to an animal and measuring inhibition of the function of the DGKε gene product in the animal, wherein a test compound which inhibits the function of the DGKε gene product is a potential agent for treating disorders selected from the group consisting of seizures, neurodegenerative disorders, and ischemic damage. In such methods, the function of the DGKε gene product in the animal to be measured is the enzymatic conversion of 20:4-DAG to 20:4-PA Some potential agents function by interfering with the binding of 20:4-DAG to DGKε.

From these methods of screening for DGK-inhibiting compounds come methods of inducing resistance to disorders selected from the group consisting of seizures, neurodegenerative disorders, and ischemic damage in a mammal. These methods include the steps of administering a compound that inhibits DGKε activity to the mammal. The compound may be selected by a method comprising the steps of contacting a cell with a test compound and measuring the inhibition of the function of the DGKε gene product in the cell. The cell used expresses or over-expresses a DGKε gene product. A test compound that inhibits the function of the DGKε gene product is a potential agent for treating disorders selected from the group consisting of seizures, neurodegenerative disorders, and ischemic damage. The compound may alternatively be selected by a method comprising the steps of administering a test compound to an animal and measuring inhibition of the function of the DGKε gene product in the animal. A test compound that inhibits the function of the DGKε gene product is a potential agent for treating disorders selected from the group consisting of seizures, neurodegenerative disorders, and ischemic damage.

The invention further includes a transgenic nonhuman animal whose germ cells and somatic cells contain at least one chromosome comprising a disruption to the endogenous DGKε gene. Some such animals may have a disruption resulting in a lack of expression of the DGKε gene product. In some cases, this disruption results from the insertion of a selectable marker gene sequence or other heterologous sequence into the genome by homologous recombination. Finally, the invention may simply comprise a cell derived from the transgenic nonhuman animal just described.

Although DGKε is the sole cloned mammalian DGK displaying high selectivity for 20:4 lipids, it is further shown herein to be possible that the function of DGKε in vivo can be compensated, at least in part, by other DGKs when DGKε is inactivated. Deficiency of DGKε selective for 20:4-DAG allowed the identification of synaptic signaling activated during epileptogenesis and contributing to seizure development. The genetic approach used herein demonstrates avenues for exploration of inositol lipid signaling, critical in generating potent messengers at the synapse.

### SUMMARY OF DRAWINGS

A more particular description of the invention briefly described above will be rendered by reference to the appended figures. These figures only provide information concerning typical embodiments of the invention and are not therefore to be considered limiting of its scope.

FIG. 1 shows the results of a Northern blot analysis of the distribution of DGKε mRNA in murine tissues;

FIGS. 2A-H show the results of an in situ hybridization analysis of the distribution of DGKε in mouse brain;

FIG. 3A illustrates the strategy for accomplishing a targeted disruption of the DGKε gene in the R1 EX cell line and C57/BL6 mice; FIG. 3B is a Southern blot analysis of selected ES cell lines; and FIG. 3C shows a Southern blot analysis of wild-type and mutant DNA extracts;

FIG. 4 is a graph summarizing the behavioral responses of wild-type, heterozygous, and homozygous DGKε mice to electroconvulsive shock;

FIG. 5 contains graphs illustrating differences in the electroconvulsive shock-induced degradation of PIP2 in DGKε−/− mice;

FIG. 6 contains graphs illustrating the electroconvulsive shock-induced accumulation of free fatty acids and DAG in wild-type and DGKε−/− mice; and

FIGS. 7A and 7B are charts summarizing the reduced long-term potentiation in hippocampal perforant path-dentate gyrus neurons in DGKε-deficient mice.

### DETAILED DESCRIPTION OF THE INVENTION

The present invention relates to DAG kinases, a family of enzymes considered to be a promising target for various therapies due to the signaling properties of the substrates of many of the enzymes and of their reaction products as well. Specifically, the invention relates to the characterization of murine DGKε and methods of screening compounds to isolate those capable of modulating DGKε activity. Further, the invention focuses on providing neuroprotection for applications such as treating disorders selected from the group consisting of seizures, neurodegenerative disorders, and ischemic damage by administering the compounds located in the screens. Because of the influence DGKs exert over the intracellular concentrations of DAG and PA, control of their activity provided by such compounds and through such methods is essential.

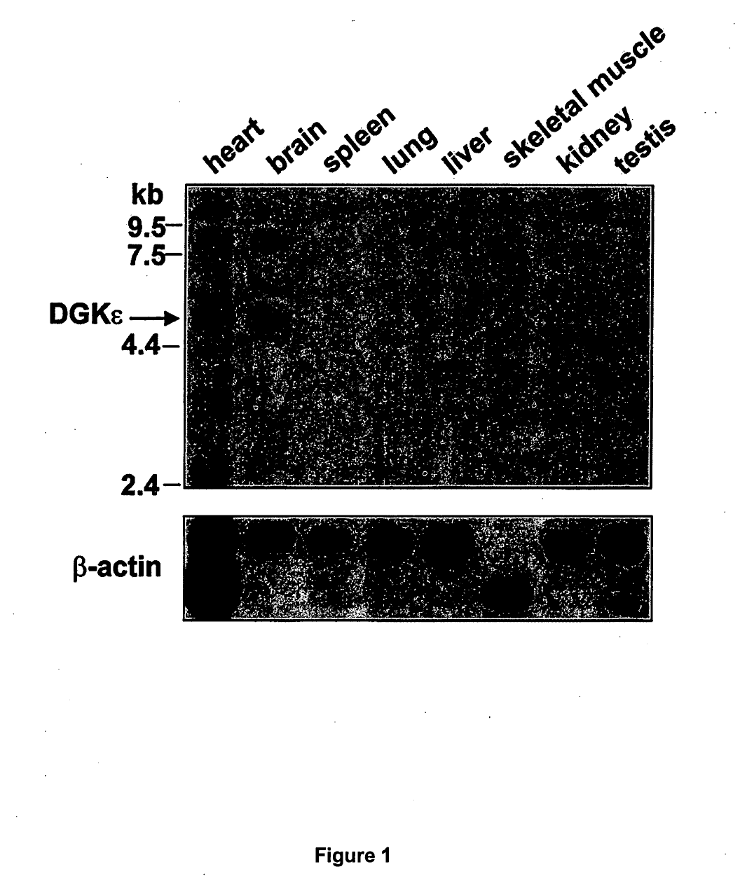

Turning now to the figures, FIG. 1 shows the distribution of DGKε in murine tissues as determined by Northern blot analysis. In the present invention, brain and heart tissues are shown to have the highest constitutive expression levels of 5- and 8-kb DGKε, while testis tissues show only the 5-kb band. Other tissues screened include spleen, lung, liver, skeletal muscle, and kidney.

In the present invention, the probe used was prepared from the coding region of the DGK gene. Using this coding-region probe, the specific mRNA band of human DGKε was also detected in brain and heart when hybridized in tissue Northern blotting. This difference may be caused by distinct specificity of probes used. Alternatively, this difference may reflect the involvement of murine and human DGKε in different signaling pathways.

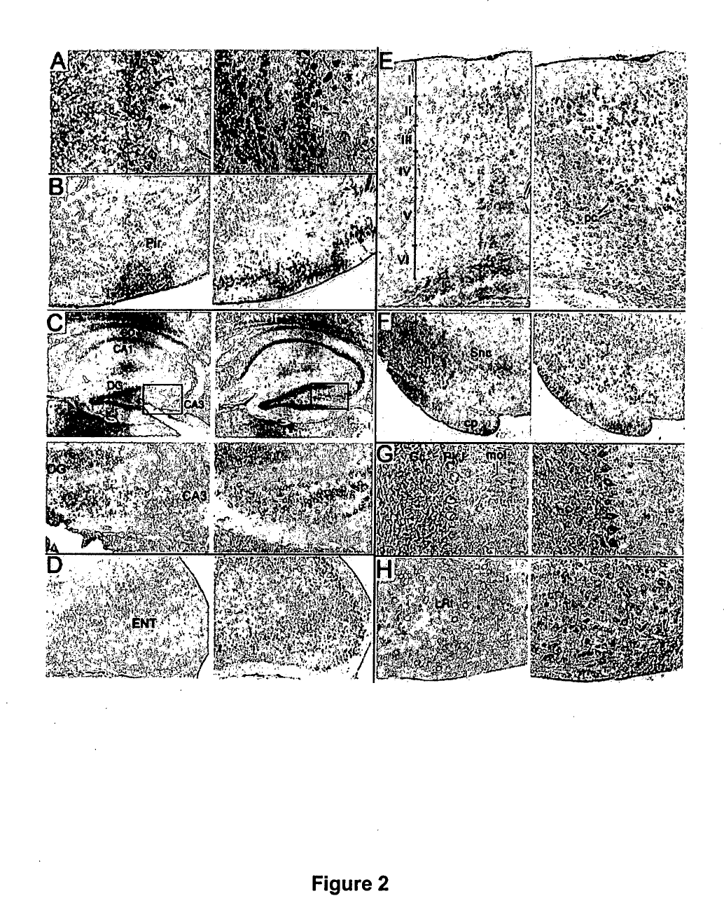

FIG. 2 shows the distribution of DGKε in the mouse brain as measured by in situ hybridization. Adjacent sagittal sections were hybridized with sense (Left) or antisense (Right) digozigenin-labeled probe prepared from a 0.9-kb EcoRI fragment of murine DGKε. (A) staining of the olfactory bulb was most notable in mitral cells (MC; x 150) and to a lesser extent in the granular cells. (B) Staining was notable in the piriform cortex (Pir; x60) but was not prevalent in adjacent cortical structures including the insular cortex (data not shown). (C) In the hippocampus (x40, and boxed regions at x160, Lower), an intense signal was visible in the pyramidal cells of CA3, while only weak staining of dentate granular (DG) cells was observed. Pyramidal cells of CA1 were labeled throughout, but no signal was seen in the stratum oriens (so), and only inconsistent staining of cells was observed in other hippocampal regions. (D) A prominent signal for DGKε RNA was detected in the entorhinal cortex (ENT; x60), and especially cells of the outer layers. (E) Staining of the medial occipital (neo) cortex (x80) was observed in all layers, but was most prominent in the pyramidal cells (pc) of layer 5.

Definition of the layers was based upon adjacent sections counterstained with Giemsa (data no shown). No staining of cells over background in the internal capsule (ic) was detected. Staining of cells in the thalamus or in structures of the basal ganglia was occasional or absent, except in F (x60) for the substantia nigra reticulata (Snr). Staining of cells in the substantia nigra compacta (Snc) was present but inconsistent. The cerebral peduncle (cp) is identified. (G) In the cerebellum (x150), staining was most intense in Purkinje cells (PKj) but could be identified above background in cerebellar granular cells (Gc) and cells of the layer molecular (mol). Staining of cells throughout the hindbrain and Pons was observed, including in the trigeminal nuclei and the superior olive (data not shown). (H) Staining was particularly intense in the lateral reticular nucleus (LRt; x150).

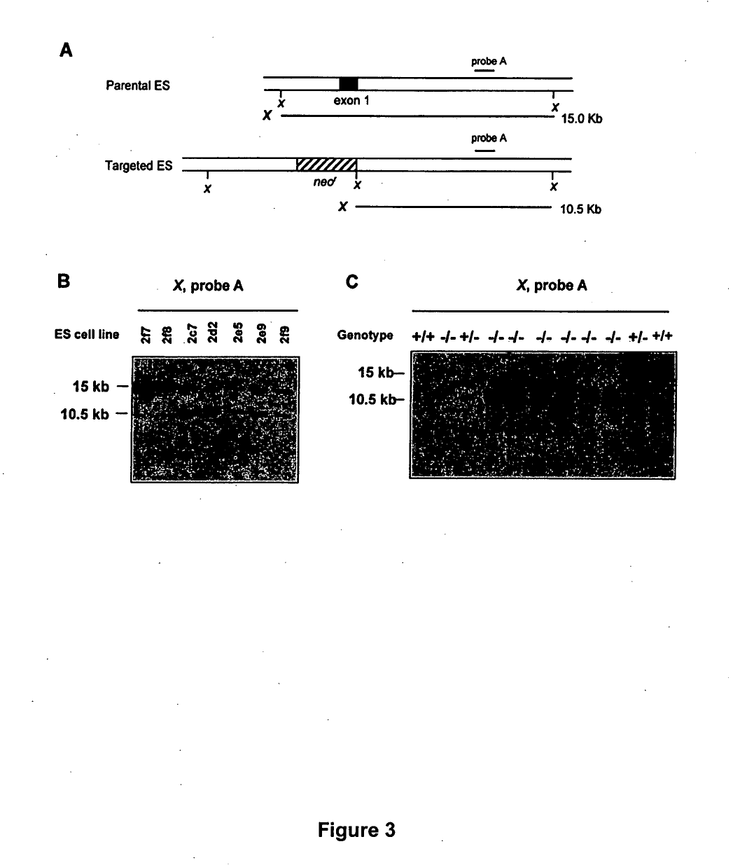

As portrayed visually in FIG. 3, mice deficient in DGKε (designated “DGKε−/− mice”) were generated using the gene targeting methods of Example 2, discussed below. FIG. 3 lays out a strategy for the targeted disruption of the DGKε gene in the R1 EX cell line and C57/BL6 mice. First, in FIG. 3A, parental and targeted DNA fragments after digestion with Xbal ES cell lines are shown. In FIG. 3B, a Southern blot analysis of selected ES cell lines is shown. Untargeted cell lines were 2f7 and 2f8. Targeted cell lines were 2c7, 2d2, 2e5, 2e9, and 2f9. Finally, FIG. 3C shows a Southern blot analysis of wild-type and heterozygous mutants taken from tail DNA extracts. The 15-kb band is wild type, and the 10.5-kb band is the targeted deletion of DGKε.

These mice were produced in order to define the significance of the 20:4-inositol lipids in seizures and LTP. Studies of these mice showed this cycle to be down-regulated, resulting in a reduced ECS-induced accumulation of 20:4-DAG. Moreover, DGKε−/− mice were more resistant to ECS and displayed attenuated LTP in perforant path-dentate granular cell synapses. These findings support the notion that 20:4-inositol lipid signaling is involved in neural responses during seizures and in hippocampal synaptic plasticity.

First, since ECS and ischemia activate the PLC-mediated release of 20:4-DAG, it was investigated whether deficiency of DGKε, which terminates 20:4-DAG signals, would affect the seizure response to ECS. Reddy & Bazan, J. Neurosci. Res., 18, 449-455 (1987); Aveldano & Bazan, J. Neurochem., 25, 919-920 (1975); Aveldago de Caldironi & Bazan, Neurochem. Res., 4, 213-221 (1979). It was found that male and female DGKε-deficient mice were more resistant to ECS, displaying shorter tonic seizures and faster recovery than DGKε+/+ mice. This behavioral response was paralleled by lower degradation of brain PIP2 and lower accumulation of DAG and FFA after ECS. Moreover, DGKε−/− mice recovered DAG basal levels within 1 minute after ECS, but a more sustained accumulation was observed in DGKε+/+ mice.

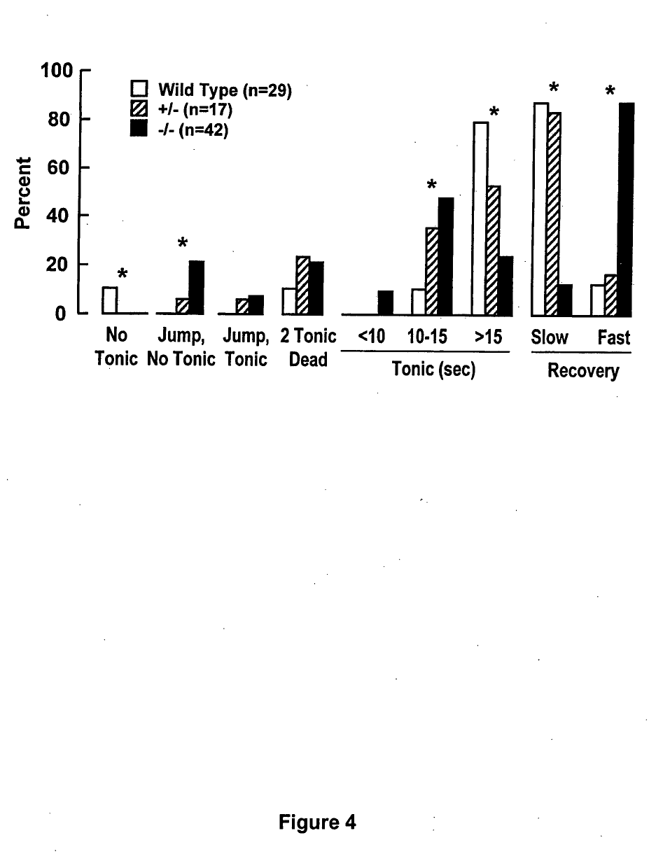

The results of these ECS studies are summarized in FIG. 4, which contains the behavioral responses of ECS-induced seizures in wild-type, heterozygous, and homozygous DGKε mice. Wild-type mice display a tonic seizure lasting 15-20 seconds, followed by clonic seizure, remaining lying on their side and recovering their posture by 3-4 minutes after ECS (slow recovery). The X2 test was used for the statistical analysis of the data. In FIG. 4, an asterisk (*) denotes significant differences for all three phenotypes in the frequency of the behavioral response to ECS (P<0.005).

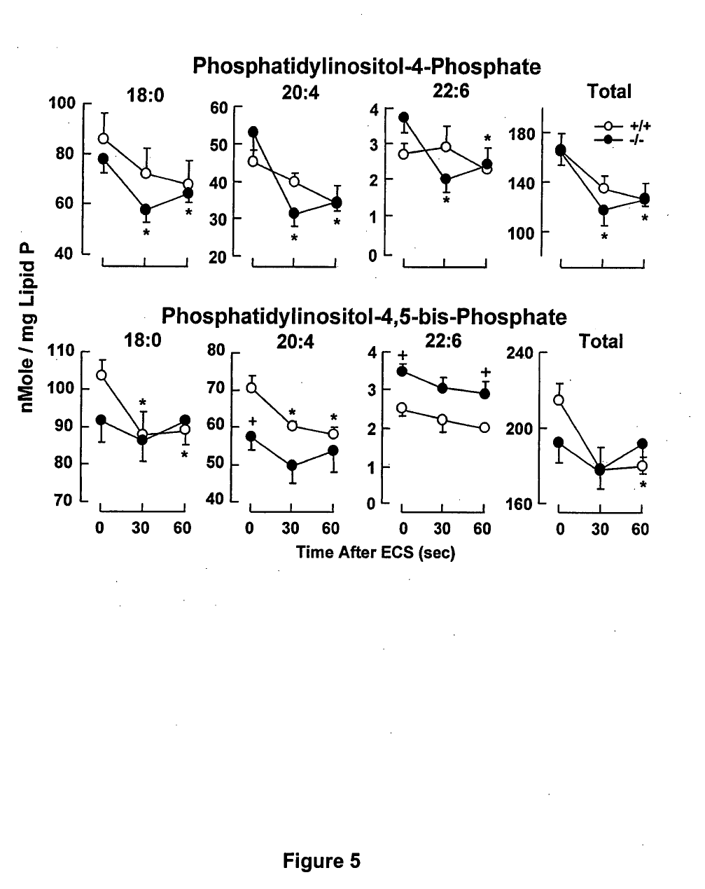

Referring now to FIG. 5, the ECS-induced degradation of PIP2 is shown to be impaired in the DGKε−/− mice. Mean values±SEM are shown in (n=4−6). Statistically significant differences (Student's t test, P<0.05) are indicated: *, with respect to control (0 time); +, DGKε−/− mice vs. DEKε+/+ mice. Although resting levels of PIP and PIP2 were similar in cerebral cortex from DGKε−/− and DGKε+/+ mice, some changes were detected. Neuronal PPI is maintained by de novo synthesis via PA, whereas the DAG- DGKε pathway contributes to their resynthesis after synaptic activity-induced PIP2 degradation. Activation of the mGluRs regulates the operation of this cycle through PLC. In DGKε−/− mice, only 20:4-PIP2 displayed decreased resting levels. Therefore, despite the deficiency in DGKε selective for 20:4-DAG phosphorylation, other DAG kinase(s) and/or the de novo synthesis pathway may partly compensate to generate 20:4-PA that, in turn, is channeled to inositol lipids.

Moreover, DGKε−/− mice did not show enrichment in 20:4-DAG and only 31% of the animals displayed higher resting levels of total DAG as compared with DGKε+/+ mice. It appears likely that the resting 20:4-DAG pool of the PPI pathway is very small compared with the pool of 20:4-DAG linked to the turnover of other phospholipids and, therefore, changes may be masked by the size of the total DAG pool.

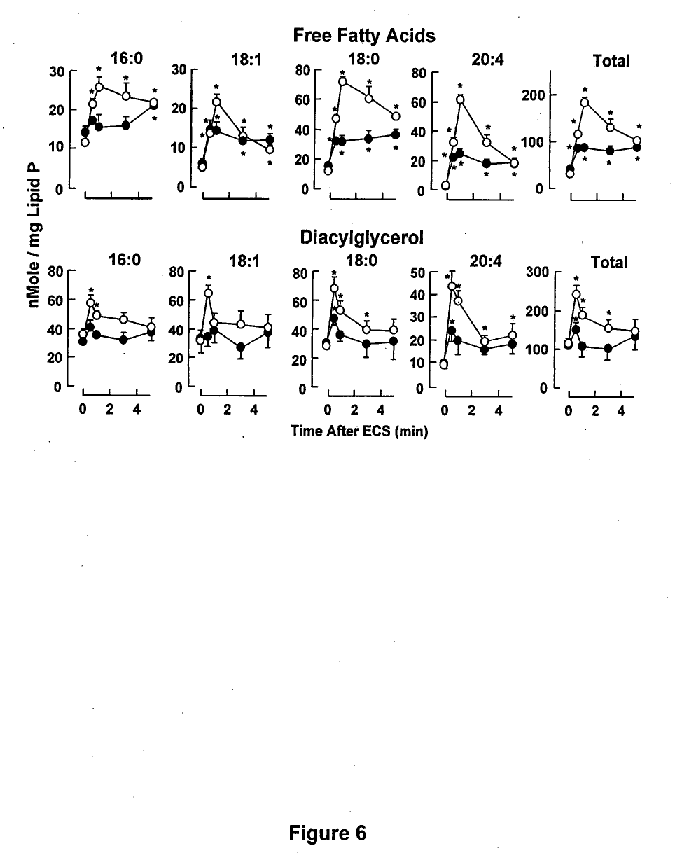

The ECS-induced 18:0- and 20:4-DAG accumulation in the cerebral cortex by 30 seconds was lower in DGKε−/− mice than in DGKε+/+ mice. However, the removal of DAG showed similar kinetics to that in DGKε+/+ mice, suggesting that, even after stimulation, other DGK(s) may compensate for the DGKε deficiency. The DAG pathway is terminated by DGK through its phosphorylation to PA and/or by DAG lipases with the generation of FFA. Bazan et al., Prog. Brain Res., 96, 247-257 (1993). However, the rapid removal of DAG in DGKε−/− mice was not paralleled by a further increase in FFA. Without being limited to any one theory, this appears to support the belief that DGKs other than DGKε, rather than DAG lipases, are involved in the efficient removal of DAG after ECS in DGKε−/− mice. FIG. 6 portrays the ECS-induced accumulation of FFA and DAG in wild-type and DGKε−/− mice. Mean values±SEM are shown. Basal values (time 0) are the average of 16 samples. Other time points are from 5-8 individual samples. In this figure, an asterisk (*) denotes values significantly different from basal (P<0.05, Student's t test).

Interestingly, DGKe−/− mice displayed very low PIP2 degradation after ECS, suggesting impairments in mGluR function linked to G proteins and PLC activation. It appears possible that to cope with the deficiency of the DGKε, adaptive and/or compensatory changes are developed. This may include a persistent PKC binding to the membrane in close association with the mGluR-G protein-PLC complex, where 20:4-DAG accumulates, because PKC controls the PPI cycle by feedback inhibition of PLC. Nishizuka, FASEB J., 9, 489-496 (1995). This sustained translocation will inhibit the PPI-PLC signaling, as reflected in the low ECS-induced 20:4-DAG release.

In DGKε−/− mice, a significant decrease of 18:0- and 20:4-PIP not observed in DGKε+/+ mice took place after ECS. Because stimulation activates degradation and resynthesis of inositol lipids, the levels of PIP and PIP2 reflect the balance of these two pathways. In DGKε+/+ mice, degradation of PIP2 occurs at a faster rate than its replenishment from PIP by PIP 4-phosphate 5-kinase, while PIP is being replenished through the DAG-PA-PI pathway. This results in a decrease of PIP2 and no detectable changes in PIP. In DGKε−/− mice, the decrease observed in PIP may indicate its active phosphorylation to PIP2 and its slower replenishment from the DAG-DGKε−/− pathway. Because type I PIP 4-phosphate 5-kinase ε is highly expressed in the brain and greatly stimulated by PA, deficiency of DGKε will likely not force PIP2 resynthesis. Ishihara et al., J. Biol. Chem., 273, 8741-8748 (1998); Anderson et al., J. Biol. Chem., 274, 9907-9910 (1999). Therefore, other metabolic dysfunctions to be considered in DGKε deficiency are the PIP degradation by PLC and/or its dephosphorylation to phosphatidylinositol.

In DGKε+/+ mice, there was also accumulation of 16:0- and 18:1-DAG after ECS, while levels of 16:0- and 18:1-PIP2 remained unchanged (data not shown). These changes are consistent with degradation of other phospholipids through a PLD pathway that contributes to the sustained accumulation of DAG. Bazan & Allan, J. Neurotrauma, 12, 791-814 (1995). In DGKε−/− mice, however, no change in 16:0- and 18:1-DAG was observed. Thus, the disruption of the inositol lipid cycle and therefore, the lack of DAG-stimulated PKC-mediated activation of PLD may lead to the loss of PLD response to stimulation. Exton, Biochim. Biophys. Acta, 1439, 121-131 (1999). These results suggest impairments in multiple signaling pathway response to ECS that, in turn, may contribute to the observed fast recovery of DGKε−/− mice from tonic-clonic seizures.

The magnitude of free 20:4 and other FFA accumulation after ECS in DGKε−/− mice compared with DGKε+/+ mice unveiled potential alterations in the calcium-dependent cPLA2 pathway. Degradation of 18:0- and 20:4-DAG generated from PIP2 after ECS may contribute to the FFA pool. Bazan, J. Neurotrauma, 12, 791-814 (1995). However, the release of 20:4 after ECS, when PIP2 levels start to recover, implicates the activation of cPLA2 targeting with high selectivity of 20:4-phospholipids. Chen et al., J. Neurophysiol., 85, 384-390 (2001).

NMDA receptor activation leads to calcium influx and cPLA2 activation. Bazan et al., J. Neurotrauma, 12, 791-814 (1995). The intracellular mobilization of calcium by IP3 also contributes to sustained activation of cPLA2. The cPLA2 signaling has a profound impact in responses to stimulation because 20:4 by itself, and eicosanoids generated by COX-1-COX-2, are involved in the modulation of synaptic activity. Bazan et al., J. Neurotrauma, 12, 791-814 (1995). Moreover, cPLA2 generates lyso-PAF, the precursor of PAF, a neuromodulator of LTP that stimulates glutamate release from presynaptic terminals and activates transcription of genes. Kato et al., Nature, (London) 367, 175-179 (1994); Clark et al., Neuron, 9, 1211-1216 (1992); Bazan et al., Prog. Brain Res., 96, 247-257 (1993); Marcheselli et al., J. Neurosci, 37, 54-61 (1994); Squinto et al. J. Neurosci Res., 24, 558-566 (1989). In cultures of hippocampal neurons from DGKε−/− mice, glutamate stimulation resulted in lower accumulation of intracellular Ca2+ compared with cells from DGKε+/+ mice (unpublished results). In this context, it is relevant that sustained increase in intracellular Ca2+ appears to be required for cPLA2 translocation to the membrane and full enzymatic activity. Hirabayashi et al., J. Biol. Chem., 274, 5163-5169 (1999). Decreased activity of cPLA2 and consequently lower release of 20:4 and PAF production in DGKε−/− mice are also supported by the observation that LTP is attenuated in perforant path-dentate granular cell synapses.

In summary, changes in lipid messengers generated in the cortex of DGKε−/− mice after one ECS were complex and suggestive of alterations in different signaling pathways (i.e., PLA2, PLC, and PLD) triggered by the deficiency in DGKε. The lack of the DGKε should lead to a higher accumulation of 20:4-DAG after ECS compared with DGK+/+ mice. Instead, changes were of much lower magnitude as a result of lower production of 20:4-DAG through PIP2-PLC, and reflected in the lower changes detected in 20:4-PIP2 compared with DGK+/+ mice. Deficiency in the PLA2 and PLC signaling by ECS leads to impaired release of messengers (i.e., 20:4, eicosanoids, PAF, DAG). These messengers are involved in the potentiation of excitatory neurotransmission favoring further glutamate release and a more efficient and sustained glutamate signaling in postsynaptic neurons. Bazan et al., J. Neurotrauma, 12, 791-814 (1995). Thus, the observed changes may underlie the higher resistance to seizure and faster recovery observed in DGK−/− mice.

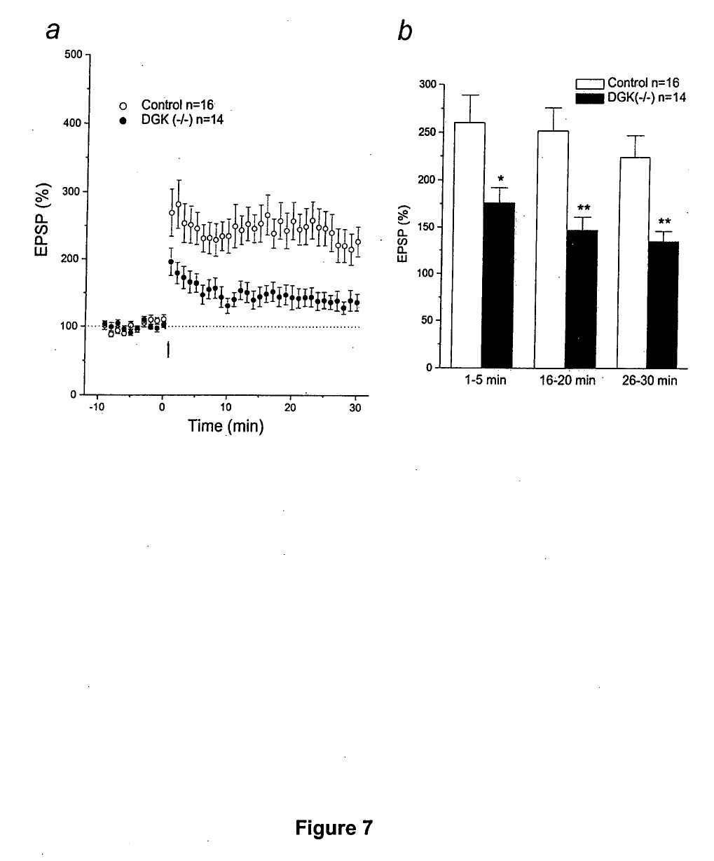

HFS-induced LTP is also shown to be reduced in perforant path-dentate granular cell synapses in DGKε-deficient mice compared with that of wild-type mice. In FIG. 7, reduced LTP in hippocampal perforant path-dentate gyrus neurons of mice deficient in DGKε is shown. FIG. 7A shows the time course and extent of LTP induction after HFS (designated by the arrow) in control and DGKε−/− mice. Excitatory Postsynaptic Potentials (“EPSP”) amplitude was normalized as percent of average baseline EPSP amplitude. FIG. 7B shows the mean potentiation of EPSP calculated by the average EPSP amplitude and 1-5 minutes, 16-20 minutes, and 26-30 minutes after HFS plotted as percent of baseline. Data are mean±SEM. *, P<0.05; **, P<0.01.

These mice displayed lower release of 20:4 induced by ECS, suggestive of lower cPLA2 activity necessary for PAF synthesis. The attenuation of LTP in DGK−/− mice may, therefore, be the consequence of diminished PAF synthesis directly involved in modulating excitatory synaptic activity. Kato et al., Nature, (London) 367, 175-179 (1994); Clark et al., Neuron, 9, 1211-1216 (1992). However, low production of other 20:4-inositol lipid-derived signaling molecules also may contribute to alterations in synaptic plasticity.

In addition, the hippocampal dentate gyrus is of significance for learning and memory and for epileptogenesis. Thus, alterations in the 20:4-inositol lipid cycle take place in limbic structures that display high DGKε expression and may contribute to synaptic dysfunction and pathology.

Thus, as discussed above, nucleic acids that encode DGKε can also be used to generate either transgenic animals or “knock out” animals that are useful in the development and screening of therapeutically useful reagents. A transgenic animal (e.g., a mouse or rat) is an animal having cells that contain a transgene, which transgene was introduced into the animal or an ancestor of the animal at a prenatal. e.g., an embryonic stage. A transgene is a DNA that is integrated into the genome of a cell from which a transgenic animal develops. In one embodiment, cDNA encoding DGKε or an appropriate sequence thereof can be used to clone genomic DNA encoding DGKε in accordance with established techniques and the genomic sequences used to generate transgenic animals that contain cells which express DNA encoding DGKε. Methods for generating transgenic animals, particularly animals such as mice or rats, have become conventional in the art and are described, for example, in U.S. Pat. Nos. 4,736,866 and 4,870,009.

Typically, in such methods, particular cells would be targeted for DGKε transgene incorporation with tissue-specific enhancers. Transgenic animals that include a copy of a transgene encoding DGKε introduced into the germ line of the animal at an embryonic stage can be used to examine the effect of increased expression of DNA encoding DGKε. Such animals can be used as tester animals for reagents thought to confer protection from, for example, pathological conditions associated with DGKε overexpression. In accordance with this facet of the invention, an animal is treated with the reagent and a reduced incidence of the pathological condition, compared to untreated animals bearing the transgene, would indicate a potential therapeutic intervention for the pathological condition.

Alternatively, non-human homologues of DGKε can be used to construct an DGKε “knock out” animal which has a defective or altered gene encoding DGKε as a result of homologous recombination between the endogenous gene encoding DGKε and altered genomic DNA encoding DGKε introduced into an embryonic cell of the animal. For example, cDNA encoding DGKε can be used to clone genomic DNA encoding DGKε in accordance with established techniques. A portion of the genomic DNA encoding DGKε can be deleted or replaced with another gene, such as a gene encoding a selectable marker that can be used to monitor integration. Typically, several kilobases of unaltered flanking DNA (both at the 5′ and 3′ ends) are included in the vector (see e.g., Thomas and Capecchi, Cell. 51:503 (1987) for a description of homologous recombination vectors). The vector is introduced into an embryonic stem cell line (e.g., by electroporation) and cells in which the introduced DNA has homologously recombined with the endogenous DNA are selected (see e.g., Li et al., Cell, 69:915 (1992)). The selected cells are then injected into a blastocyst of an animal (e.g., a mouse or rat) to form aggregation chimeras (see e.g., Bradley, in Teratocarcinomas and Embryonic Stem Cells: A Practical Approach, E. J. Robertson. ed., IRL, Oxford. 1987, pp. 13-152). A chimeric embryo can then be implanted into a suitable pseudopregnant female foster animal and the embryo brought to term to create a “knock out” animal. Progeny harboring the homologously recombined DNA in their germ cells can be identified by standard techniques and used to breed animals in which all cells of the animal contain the homologously recombined DNA. Knockout animals can be characterized for instance, for their ability to defend against certain pathological conditions and for their development of pathological conditions due to absence of the Apaf-1 polypeptide, including for example, non-regulated growth of cells and/or development of tumors.

The DGKε protein is useful in assays for identifying therapeutically active molecules that modulate apoptosis. Specifically, compounds that either inhibit DGKε or enhance DGKε can be conveniently identified by these screening methods. Molecules inhibiting DGKε are useful to cause neuroprotection, and, more specifically, prevent disorders selected from the group consisting of seizures, neurodegenerative disorders, and ischemic damage. Molecules enhancing or promoting DGKε are useful, for example, in neurological and seizure-related research.

Assay of candidate compounds able to competitively compete with DAG for specific binding to DGKε provides for high-throughput screening of chemical libraries, and is particularly useful for screening small molecule drug candidates. Small molecules, usually less than 10 K molecules weight, are desirable as therapeutics since they are more likely to be permeable to cells, are less susceptible to degradation by cells, and are not as apt to elicit an immune response as larger oligonucleotides. Small molecules include, but are not limited to, synthetic organic or inorganic compounds. Many pharmaceutical companies have extensive libraries of such molecules, which can be conveniently screened by assessing binding to DGKε. As part of these assays, measurement of DGKε inhibition may be conducted in a variety of ways known in the art, including those discussed in the following Examples.

### EXAMPLES

Materials and Methods

The following materials and methods were practiced in the examples of the invention that follow.

Isolation of Murine DGK cDNA and Genomic Clones

A human DGK cDNA fragment was used as a probe to screen a murine testis λgt 11 cDNA library (CLONTECH). One 1.1-kb clone with nearly the full-length coding sequence was used to screen a murine 129SVJ Fix II genomic library (Stratagene) as described in Tang et al., Gene, 239, 185-92 (1999). Positive clones were subcloned into pBluescript II SK (+), and the genomic organization was analyzed by a combination of restriction mapping, Southern blotting, subcloning, and automated sequencing. Tang et al., Gene, 239, 185-92 (1999). A full-length cDNA clone was generated by using PCR from the genomic clone and then it was subcloned into pcDNA3 (Invitrogen) for expression of the protein in mammalian cells.

Tissue Culture, Tranfection, and Analysis of DGK Activity

COS-7 cells were first cultured and transfected as in Buntig et al., J. Biol. Chem., 271, 10230-10236 (1996). Next, in vitro DGK assays using the cell lysates were performed. Tang et al., J. Biol. Chem., 271, 10237-10241 (1996).

Analysis of Multiple-Tissue Northern Blot

A murine multiple-tissue Northern blot (CLONTECH) was probed with a 32P-labeled fragment of murine DGK corresponding to nucleotides 532-1580 by using ExpressHyb (CLONTECH) according to the manufacturer's protocol.

Histology and in Situ Hybridization

Probes for in situ hybridization corresponded to nucleotides 532-1580 of murine DGK and nucleotides 192-791 of murine glyceraldehydes-3-phosphate dehydrogenase. Sections were deparaffinized, rehydrated, and incubated at room temperature for 30 minutes with proteinase K (6 μg/ml) and then for 10 minutes in 0.25% acetic anhydride/0.1 M triethanolamine. Sections were dehydrated and hybridized for 16 h at 58° C. with digozigenin-labeled sense or antisense RNA probes (1 μg/ml) in a solution of 10% dextran sulfate, 50% formamide, 100 mM DTT, 0.3 M NaCl, 5 mM EDTA, 1× Denhardt's solution (0.02% polyvinylpyrrolidone/0.02% BSA), yeast tRNA (500 μg/ml), and 20 mM Tris-HCl (pH 7.5). Sections were then washed for 1 h at room temperature in 4×SSC/10 mM DTT and 30 minutes at 58° C. in 50% formamide/1×SSC/10 mM DTT. After digestion with RNase A (10 μg/Ml) for 30 minutes at 37° C., sections were washed 15 minutes in 2×SSC, 15 minutes in 0.1×SSC, and then 2 minutes in buffer 1 (100 mM maleic acid/150 mM NaCl, pH 7.5). Sections then were incubated at room temperature for 30 minutes in buffer 1/10% normal sheep serum, then with anti-digoxigenen alkaline phosphatase conjugate (1:500 dilution in bugger 1/10% normal sheep serum/0.3% Triton X-100) for 2 h, and were washed for three 10-minute periods in buffer 1 and for 2 minutes in buffer 3 (100 mM NaCl/0 mM MgCl2/100 mM Tris-HCl, pH 9.5). Signals were detected after incubation in nitroblue tetrazolium (338 μg/Ml)/5-bromo-4-chloro-3-indolyl phosphate (175 μg/Ml) in buffer 3 for several hours to 1 day.

Construction of the DGK Targeting Vector and Screen for Targeted Embryonic Stem (ES) Cells

A DGK targeting vector was produced by replacing exon 1 of the DGK gene with a neomycin-resistance (neor) cassette, which provided for positive selection. This and flanking genomic DNAs were subcloned into the phage targeting vector MDASHII-2TK254 (Kirk Thomas, University of Utah), placing it between two thymidine kinase genes for negative selection. The linearized vector was electroporated into R1 ES cells, which then were screened by positive-negative selection. Thomas et al., Nature, (London) 346, 847-850 (1990). To screen ES cell clones for homologous recombination, DNA from selected EX cell lines was digested with XbaI and subjected to Southern blot analysis using probe A. Tang et al., Gene, 239, 185-92 (1999). Of 72 selected clones, 17 (23%) contained the targeted allele. The homologous recombinant clones (with a 10.5-kb, rather than a 15.0-kb, fragment) were then injected into C57/BL6 blastocysts that were injected into uteri of pseudopregnant C57/BL6 females. Resulting male chimeric mice were back-crossed with BL/6 females and heterozygous mutants were identified by genomic Southern blotting and PCR by using tail DNA as described below.

Genotyping by PCR and Southern Blotting

Genotyping was performed at the time of birth and again the day of the experiment 2-3 months later. Mouse tail (1-2 cm) was digested [5 mM EDTA/200 mM NaCl/100 mM Tris-HCl, pH 8.0/0.2% SDS/proteinase K (0.5 mg/ml)/RNase A (12.5 g/ml)] overnight in a water bath at 55° C. The DNA was extracted with phenol/chloroform/isoamyl alcohol, 25:24:1 (vol/vol), washed twice in chloroform/isoamyl alcohol, 26:4 (vol/vol), and precipitated with 1:1 isopropanol. Pellets were resuspended in Tris/EDTA (pH 8.0) and heated for 2 h at 65° C. Southern blotting was performed as described above. Each PCR mixture contained 0.5 μl of DNA, 1×Pfu Turbo buffer, all four dNTPs (each at η2 mM), 2.5 μM forward and reverse primers, 1% Tween-20, water, and Pfu Turbo DNA polymerase (2.5 unit) in a total volume of 25 μl. A forward primer from a region on the 5′ side of exon 1 (5′-AGAGAGGCACGGGCGAGGCTC-3′: SEQ ID NO: 1) and a reverse primer in exon 1 (5′-GCGCGACCGCTGCAGGCTACA-3′: SEQ ID NO: 2) were used to amplify the wild-type allele. The same forward primer and a reverse primer for the neor cassette (5′-CAGGACGTTGGGGCACCGCCT-3′: SEQ ID NO: 3) were used to identify the mutant allele. Homozygous product size was 242 bp and the wild-type product size was 344 bp. Reactions were carried out at 94° C. for 5 minutes, 94° C. for 1.0 minute, 65° C. for 1.5 minutes, and 72° C. for 1.5 minutes for 35 cycles and then 72° C. for 5 minutes.

Electroconvulsive Shock

Two- to 3-month-old mice (20-25 g, male/female) were implanted with two platinum electrodes under the scalp (parallel, 1 cm apart). A single stimulation train of square pulses was delivered at 50 V dc and 0.5-msec pulse duration with a frequency of 100 pulses per second, a train duration of 200 msec, and a train rate of 0.750 train per second. Marcheselli et al., J. Neurosci., 37, 54-61 (1994). This stimulation evoked, in wild-type mice, a tonic seizure lasting 15-20 seconds, followed by a clonic seizure. Differences in the behavioral response to ECS among wild-type, heterozygous (+/−), and homozygous (−/−) DGKε mice are summarized in FIG. 4.

Lipid Analysis

Mice were killed by high-frequency head-focused microwave irradiation, their heads were cooled quickly in ice-cold water, and their brains were dissected. Lipids from the right cerebral cortex were extracted with hexane/isopropanol, 3:2 (vol/vol), for the analysis of free fatty acids (FFA) and DAG. Polyphosphoinositides (PPI) were extracted from the left cerebral cortex with acidified chloroform/methanol. Lipid classes by TLC were isolated and their acyl groups were analyzed by gas liquid chromatography. Rodriguez et al., J. Biol. Chem., 272, 10491-10497 (1997).

Electrophysiological Recordings

Hippocampal slices were prepared from either sex of DGK-deficient mice and age-matched control mice, as described in Chen et al., J. Neurophysiol., 85, 384-390 (2001), and individual dentate granular cells were visualized with a Zeiss Axioskop microscope,

Whole-cell patch-clamp recordings were made with an Axoc-lamp-2B patch-clamp amplifier in bridge mode as described in Chen et al., J. Neurophysiol., 85, 384-390 (2001). Data were acquired (25 kHz, filtered at 1 kHz) by using a DigiData 1200 interface and PCLAMP 7.01 software (Axon Instruments, Foster City, Calif.). Excitatory postsynaptic potentials (“EPSPs”) in response to stimulation of the perforant path were recorded at a frequency of 0.005 Hz and the amplitude range of the evoked EPSPs was always adjusted to 2-6 mV (<30% threshold for generating an action potential). Long term potentiation (“LTP”) in the performant path was inducted by high-frequency stimulation (“HFS”) consisting of eight trains, each of eight pulses at 200 Hz with an intertrain interval of 2 seconds, as described in Wang et al., J. Physiol., (London) 495, 755-767 (1996). LTP was operationally defined as >20% increase above baseline for the amplitude of ESPSs from 26 to 30 minutes after HFS.

Statistical Analysis

Data are the mean±standard error of the mean (“SEM”). Statistical analysis was performed with the unpaired Student's t test. One-way analysis of the variance (“ANOVA”) with Fisher's PLSD post hoc was used for statistical comparison when appropriate. Statistical analysis of the frequency of the behavioral responses to ECS (see FIG. 4) was done with the x2 test. Differences were considered significant when P<0.05.

### Example 1

### Cloning and Characterization of Murine DGK

Because human DGK is expressed predominantly in testis, a murine testis cDNA library was screened with a human DGK cDNA fragment as a probe. A 1.1-kb partial clone was obtained and used to screen a murine genomic library. With clones obtained from this screen, the full-length cDNA was secured by PCR. Murine DGK is a 564-aa protein with a calculated molecular mass of 64 kDA. Alignment of the murine and human orthologs revealed 91% amino acid identity. Like its human counterpart, murine DGK displayed high selectivity for 20:4-DAG when compared with oleoyl-DAG (10.8±1.3-fold higher, n=3) and other DAG species (data not shown). To determine the distribution of murine DGK, a multiple tissue Northern blot was probed, revealing specific 5-kb and 8-kb bands, most highly expressed in brain and heart (FIG. 1). A 5-kb band was also apparent is testis.

In contrast, human DGK was highly expressed in testis and barely detectable in other tissues. Tang et al., J. Biol. Chem., 271, 10237-10241 (1996). However, Kohyama-koganeya et al. FEBS Lett., 409, 258-264 (1997), noted that rat DGK mRNA was also enriched in brain and heart. A subsequent reprobe of a human multiple-tissue Northern blot with a different fragment of DGK as a probe detected signals in the brain and heart (data not shown), confirming that the bands observed on the murine blot represent DGK mRNA. DGK sublocalization in murine brain tissue, by in situ hybridization, revealed the highest signals in Purkinje cells of the cerebellum, pyramidal cells of the hippocampus, mitral cells of the olfactory bulb, and neurons of the substantia nigra (FIG. 2). This distribution corresponded well with that reported for rat DGK. Kohyama-koganeya et al., FEBS Lett., 409, 258-264 (1997). Lower expression of DGK in neurons of the thalamus, superior olive, and lateral reticular nucleus was also detected.

### Example 2

### Generation of DGKε-Deficient Mice

The C1 domains of DGK were necessary for its activity, because deleting them rendered DGK inactive (data not shown). Exon 1 of the murine DGKε gene encoded the initiation methionine and the first and most of the second C1 domains, so a vector for targeted deletion that replaced this exon with a neomycin-resistance insert was designed (FIGS. 3A, 3B, 3C, and FIG. 4). Properly targeted, the deletion construct should result in a null mutation. Heterozygous mice (DGKε+/−) were viable and fertile and were intercrossed to obtain DGKε −/− mice. The genotype of the offspring was determined by Southern blotting (FIG. 3C), where targeted deletion resulted in a 10.5-kb band instead of a 15-kb band. These results were verified by a PCR screen (data not shown). A Mendelian pattern of inheritance of the targeted allele with a normal gender distribution was found, indicating that the deletion did not cause embryonic lethality. Homozygous DGKε null mice appeared normal and reproduced and behaved normally. No gross or histological abnormalities in major organs, including the brain, were found in DGKε−/− mice.

### Example 3

### Behavioral Responses to ECS in DGKε Knockout Mice

Because PPI signaling by mGluRs is stimulated by ECS, Reddy et al., J. Neurosci. Res., 18, 449-455 (1987), the behavioral response to ECS of DGKε−/− mice was studied. These mice displayed shorter tonic seizures compared with DGKε+/+ mice, with only 24% of the animals having sustained tonic seizure >15 seconds and 50% developing a 10- to 15-second tonic seizure (FIG. 4). After the clonic phase, DGKε−/− mice recovered faster than DGKε+/+ mice, within 1-3 minutes after ECS. Approximately 28% of DGKε−/− mice jumped immediately after stimulation, developing a very short tonic seizure (7-8 seconds, 21%) after a 3-second delay or no tonic seizure (7%). The length of the tonic seizure in heterozygous mice for the null allele (DGKε+/−) was intermediate between DGKε+/+ and DGKε−/− mice, whereas their recovery was very slow as observed in DGKε+/+ mice. No differences in behavioral responses to ECS were observed between males and females.

### Example 4

### DGKε−/− Mice Display Decreased ECS-Induced Polyphosphoinositides Degradation and Lower Accumulation of DAG and FREE 20:4

PPI content in cerebral cortex was unchanged in DGKε−/− mice compared with DGKε+/+ mice under resting conditions, except for a decrease of 20:4-PIP2 and a higher content of docosahexaenoic acid (22:6n-3) in PIP and PIP2 p<0.01; FIG. 5). Stearoyl (18:0)- and 20:4-PIP2, as expected, rapidly decreased in DGKε+/+ mice after ECS, but no significant changes were observed in DGKε−/− mice (FIG. 5). In contrast, 18:0- and 20:4-PIP were decreased only in DGKε−/− mice. 22:6-PIP, on the other hand, showed lower content 30 seconds and 1 minute after ECS only in DGKε−/− mice.

### Example 5

### DAG Resting Levels Varied Among DGKε −/− Mice

From the 16 DGKε −/− mice analyzed (mean±SEM, 108±21 nmol per mg of lipid phosphate), five showed relatively high content of DAG (n=10; DGK ε −/− mice, 110±15 nmol per mg of lipid phosphate DGKε −/−:217±30 nmol per mg of lipid phosphate; P<0.02) and 11 mice displayed very low DAG content (57±6 nmol per mg of lipid phosphate, P<0.02) compared with DGK ε −/− mice. However, the acyl group composition remained unchanged in all of the DGKε −/− mice studied. No differences were observed in the resting FFA pool size and composition, including free 20.4 FFA and DAG content in the cortex from DGKε −/− and DGKε+/+ mice after ECS are shown in FIG. 6. Within 30 seconds after ECS, wild-type mice displayed a 2.2-fold increase in DAG, decreasing thereafter and reaching basal values by 3 minutes. All acyl groups contributed to the transient enlargement of the DAG pool, 20:4-DAG displaying the slower recovery and remaining 2.4-fold above the basal level by 5 minutes. Accumulation of DAG was lower in DGKε −/− mice (1.4-fold by 30 seconds) recovering basal values by 1 minute after ECS. Only 18:0- and 20:4-DAG were significantly increased by 30 seconds. From the 25 DGKε −/− mice subjected to ECS, 44% did not show changes in DAG and FFA and were not included in FIG. 6. Interestingly, this group includes all mice that did not develop tonic seizures and some that showed 10- to 12-second seizures.

The FFA pool was increased in DGKε −/− mice by 3.8- and 6-fold at 30 seconds and 1 minute after ECS, respectively, decreasing thereafter but remaining 3.2-fold above the basal level by 5 minutes. In contrast, in DGKε −/− mice, FFA only reached a 2.1-fold increase by 30 seconds after ECS, remaining unchanged thereafter. All fatty acids reached values significantly lower than DGKε −/− mice after ECS, 18:0 and 20:4 displaying the greatest differences. Free 20:4 and 18:1 displayed the faster recovery toward basal values in DGKε −/− mice, but remained at the same level reached by 30 seconds after ECS in DGKε −/− mice.

### Example 6

### Induction of LTP is Attenuated in the Perforant Path Dentate Gyrus Cell Synapses of DGKε-Deficient Mice

Synaptic transmission and plasticity were examined in dentate granular cells of DGKε -deficient mice and their age-matched normal controls. There were no abnormalities in basic membrane properties, including resting membrane potential, input resistance, and action potential generation (8-10 spikes per burst) in cells from hippocampal slices from enzyme-deficient mice. As indicated in FIG. 7, however, the potentiation of EPSP amplitude by HFS was significantly reduced in cells from DGKε −/− mice (potentiation, mean±SEM, 135±11% of baseline from 26 to 30 minutes after HFS) when compared with that in the DGKε −/− mice (224±23%).

Conclusion: The present invention relates to the elucidation of the function of DAG kinase epsilon (DGKε). The DGK family of enzymes occupies a signaling crossroads since they catalyze the phosphorylation of DAG to produce PA. Both the substrate (DAG) and the product (PA) of this reaction are key factors in intracellular signaling, making the regulation of DGKε activity important to understand and control. DGKε displays selectively for 20:4-DAG and is highly expressed in different areas of the brain, including Purkinje cells in the cerebellum, hippocampal interneurons, and the Pyramidal neurons in the CA3 region of the hippocampus.

Mice with targeted disruption of the DGKε were generated. These mice showed that a DGKε deficiency affects multiple signaling including the PIP2-PLC and the cPLA2-20:4 pathways. This in turn, leads to higher resistance of neurons to seizures and attenuation of LTP. Moreover, higher resistance to ischemic neuronal damage may also occur. Although DGKε is the sole cloned mammalian DGK displaying high selectivity for 20:4 lipids, it appears from the results that it is possible that the function of DGKε in vivo can be compensated, at least in part, by other DGKs when DGKε is inactivated. Deficiency of DGKε selective for 20:4-DAG allowed the identification of synaptic signaling activated during epileptogenesis and contributing to seizure development. The genetic approach used herein demonstrates avenues for exploration of inositol lipid signaling, critical in generating potent messengers at the synapse.

The present invention may be embodied in other specific forms without departing from its structures, methods, or other essential characteristics as broadly described herein and claimed hereinafter. The described embodiments are to be considered in all respects only as illustrative, and not restrictive. The scope of the invention is, therefore, indicated by the appended claims, rather than by the foregoing description. All changes that come within the meaning and range of equivalency of the claims are to be embraced within their scope.

1. A method of inducing resistance to disorders selected from the group consisting of seizures, neurodegenerative disorders, and ischemic damage in a mammal, the method comprising: administering a compound which inhibits DGKε activity.

2. The method of claim 1, wherein the compound is selected by a method comprising the steps of:
contacting a cell with a test compound, wherein the cell expresses or over-expresses a DGKε gene product; and
measuring the inhibition of the function of the DGKε gene product in the cell, wherein a test compound which inhibits the function of the DGKε gene product is a potential agent for treating disorders selected from the group consisting of seizures, neurodegenerative disorders, and ischemic damage.

3. The method of claim 1, wherein the compound is selected by a method comprising the steps of:
administering a test compound to an animal; and
measuring inhibition of the function of the DGKε gene product in the animal, wherein a test compound which inhibits the function of the DGKε gene product is a potential agent for treating disorders selected from the group consisting of seizures, neurodegenerative disorders, and ischemic damage.
